(19) United States
(12) Patent Application Publication
(10) Pub. No.: US 2026/0308489 A1
(43) Pub. Date: Oct. 8, 2026

(54) SELF-ADHESIVE SANITARY PRODUCT COMPRISING ALKYL POLYGLYCOSIDE

(71) Applicant: HENKEL AG & CO. KGAA, Düsseldorf (DE)

(72) Inventors: Marianne SCHMELING, Korschenbroich (DE); Daniela POETHKOW, Krefeld (DE)

(21) Appl. No.: 19/483,289

(22) PCT Filed: May 21, 2024
(86) PCT No.: PCT/EP2024/063947

§ 371 Date: Nov. 11, 2025

(30) Foreign Application Priority Data
May 25, 2023 (EP) 23175476.3

(51) Int. Cl. C11D 17/00; A61L 2/23; A61L 9/05; A61L 103/40; C11D 1/14; C11D 1/22; C11D 1/66; C11D 1/72; C11D 1/94

(52) U.S. Cl. CPC C11D 17/0056; A61L 2/23; A61L 9/05; C11D 1/94; C11D 1/945; A61L 2103/40; C11D 1/143; C11D 1/22; C11D 1/662; C11D 1/72

(57) ABSTRACT

A self-adhesive sanitary product includes alkyl polyglycoside. The self-adhesive sanitary product is to be applied to the surface of a sanitary article and to clean and deodorize a sanitary article.

The invention relates to an adhesive sanitary product for cleaning and/or disinfecting and/or for releasing fragrances for sanitary articles such as toilet bowls.

WC cleaning agents in lump form have long been used in the toilet bowl under the rim (so-called rim blocks) and in the water tank (in-tank blocks or cistern blocks). The formulations usually contain surfactants, disinfectants and bleaching agents, salts, acids, complexing agents, fillers, dyes and fragrances, rinse regulators and/or further components. During production, the ingredients are usually mixed, compressed and then extruded into strands. The strands are finally cut to the desired length and optionally placed in toilet rim hangers.

Several effects can be achieved with such a WC block. The toilet bowl, for example, can be cleaned and/or disinfected. It is also possible to inhibit unpleasant odors or to use a perfume to deodorize it, for example. However, the ingredients needed for these different effects are not always compatible with one another. For example, sensitive substances such as dyes can be attacked by antimicrobial bleaching agents, for example; acids or complexing agents used to prevent limescale, rust or urine scale deposits are often not compatible with perfumes either.

In addition to WC sticks, which are positioned in a WC rim hanger under the toilet rim, self-adhesive WC cleaning products, which are stuck to the toilet bowl by the user, where they then adhere and are rinsed away by the flush water, have been available for some time now.

These sanitary products are viscous and generally pasty products that are applied directly to the surface of the sanitary article from a suitable container, where they adhere and can be rinsed off only after a relatively large number of flushes.

Due to the direct adhesion of the product to the surface of the sanitary article, it is not necessary to additionally provide containers such as the so-called “WC rim hangers”, the use of which is perceived by the consumer to be unhygienic, especially when replacing the sanitary product and when cleaning the toilet.

Such adhesive sanitary products are known from WO 99/66017. In addition to surfactants, water and fragrances, they also contain adhesion promoters and have so far generally been designed as solid gels. It is also known from WO99/66017 that solid gels can additionally comprise glycols and glycol derivatives. The known adhesive sanitary products can be applied using a suitable device, adhere to the surface of the sanitary article and are completely dissolved only after a large number of flushes.

The adhesive sanitary products known from the prior art are in the form of solid gels. A solid gel is usually a disperse system consisting of at least two components. The solid component forms a spongy, three-dimensional network, the pores of which are filled with a liquid (lyogel) or a gas (xerogel). The liquid component is thereby immobilized in the solid component. In the solid gels used as adhesive sanitary products, the liquid component is usually water. The solid component is usually formed by molecular “adhesion promoters” which have a hydrophilic part and a hydrophobic part and form the three-dimensional network, whose pores are filled with the liquid component, through suitable hydrogen bridge bonds.

Although such solid gels can indeed be considered to adhere to the basin to a satisfactory degree, these gels are perceived by consumers as being visually unappealing. One reason for this is the fact that the solid gels are partially transparent. Furthermore, the gel-like consistency of the sanitary products is perceived by consumers as unappealing. In extreme cases, a poorly soluble residue also forms on the sanitary article.

The object of the present invention is to provide a non-gel-like sanitary product which is still suitable for adhering to a smooth surface.

This object is achieved by the features of claim 1. According to the invention, a self-adhesive sanitary product comprising alkyl polyglycoside is described. In particular, the use of alkyl polyglycoside allows makes possible adhesion to a sanitary article. In particular, it is surprising that APG can facilitate the adhesion of non-gel-like products.

Within the context of the present invention, the term “self-adhesive sanitary product” is to be understood to mean that the product can be applied directly to the surface of the sanitary article where it adheres and can be rinsed off only after a large number of rinses, preferably at least 10 rinses.

Alkyl polyglycosides (APGs) are also used as surfactants. They are non-ionic surfactants and belong to sugar surfactants. They are used as a surface-active ingredient in detergents, dishwashing detergents and cleaning agents. Alkyl polyglycosides consist entirely of the renewable raw materials sugar, obtained from sugar cane and sugar beet, and fatty alcohols, obtained from palm oils. They are biodegradable and have low toxicity.

In this respect, the APG is able not only to make the sanitary product adhere to the surface, but also contributes to the cleaning performance.

The composition according to the invention is capable of maintaining the consistency of the adhesive sanitary product even over a large number of flushes so that the applied product maintains a smooth, substantially surface even at low flushing frequency. The product according to the invention also prevents the surface from drying with the formation of poorly soluble residues observed with known products.

The use of the composition according to the invention also has a positive effect on the service life of the product, since these compounds improve the dissolving behavior of the sanitary product and thus reduce the amount of perfume that needs to be used.

According to a preferred embodiment, a self-adhesive sanitary product is described in which the self-adhesive sanitary product is not a solid gel. In particular, the sanitary product is designed as a solid molding compound and thus has a visual impression that is preferred by many consumers over a partially transparent gel. The rinsing behavior of the molding compound also differs from that observed for solid gels.

According to a further preferred embodiment, the sanitary product is not transparent, i.e. it is essentially completely opaque. This is where the sanitary product differs from solid gels, which are usually at least partially transparent.

According to a preferred embodiment, a self-adhesive sanitary product is described in which the alkyl polyglycoside is present in an amount of at most 24 wt. %.

According to a preferred embodiment, a self-adhesive sanitary product is described in which the alkyl polyglycoside is present in an amount of at least 2 wt. %.

According to a preferred embodiment, a self-adhesive sanitary product is described in which the alkyl polyglycoside is present in an amount of 1 wt. % to less than 24 wt. %, preferably 2 to less than 24 wt. %, more preferably 4 wt. % to 20 wt. %, and particularly preferably from 9 to 15 wt. %.

According to a preferred embodiment, a self-adhesive sanitary product is described, comprising at least one anionic surfactant.

According to a preferred embodiment, a self-adhesive sanitary product is described in which the at least one anionic surfactant comprises an olefin sulfonate.

According to a preferred embodiment, a self-adhesive sanitary product is described in which the olefin sulfonate is present in an amount of at least 2 wt. % to 30 wt. %, preferably 7 wt. % to 15 wt. %.

According to a preferred embodiment, a self-adhesive sanitary product is described, comprising at least one further anionic surfactant.

According to a preferred embodiment, a self-adhesive sanitary product is described in which the at least one further anionic surfactant is an alkylbenzene sulfonate.

According to a preferred embodiment, a self-adhesive sanitary product is described in which the alkylbenzene sulfonate is present in an amount of at least 5 wt. % to 40 wt. %, preferably 10 wt. % to 30 wt. %.

According to a preferred embodiment, a self-adhesive sanitary product is described, comprising at least one further non-ionic surfactant.

According to a preferred embodiment, a self-adhesive sanitary product is described in which the at least one further non-ionic surfactant is a fatty alcohol ethoxylate.

According to a preferred embodiment, a self-adhesive sanitary product is described in which the alkylbenzene sulfonate is present in an amount of at least 2 wt. % to 10 wt. %.

According to a further aspect, the use of the self-adhesive sanitary product according to one of the preceding claims is described, wherein the sanitary product is applied to a surface of a sanitary article. The sanitary products according to the invention can be rinsed off the surface only after a relatively large number of flushes. The product according to the invention is an ointment-like, pasty, preferably non-gel-like, non-transparent product. The products are substantially dimensionally stable so that they do not “run down” or “drip” and thus remain on the surface. The products according to the invention can be applied in different ways.

Furthermore, the product may contain non-ionic surfactants. The molecules of the non-ionic surfactant are longer or a long-chained, substantially elongated molecules which are at least partially hydrophilic and thus comprise at least one hydrophilic residue or a hydrophilic group which interacts with water.

Preferably, the non-ionic surfactants should be unbranched molecules, in order to enable the desired network formation.

The non-ionic surfactant can be either entirely hydrophilic or partially hydrophilic and partially hydrophobic.

Organic molecules with a hydrophilic and a hydrophobic end can be used as non-ionic surfactants. Polyalkoxy groups, preferably polyethoxy groups, polypropoxy groups or polybutoxy groups, or mixed polyalkoxy groups such as poly(ethoxypropoxy) groups, can be used as hydrophilic residues. It is particularly preferred to use as the hydrophilic end a polyethoxy residue comprising between 5 and 55 ethoxy groups, preferably between 10 and 30 and particularly preferably 10 to 20 ethoxy groups.

If the non-ionic surfactants also have a hydrophobic end, straight-chain alkyl residues are particularly suitable for the hydrophobic residue, with an even number of alkyl residues in particular being particularly preferred due to their better biodegradability.

If alkyl groups are chosen as the hydrophobic residues, alkyl residues with at least twelve carbon atoms are preferred. Particularly good results were achieved with an alkyl chain length between 16 and 30 carbon atoms, in particular 16, 18, 20 and 22 carbon atoms.

As hydrophobic residues, non-ionic surfactants can also comprise alkylbenzene residues, for example dodecylbenzene or similar residues, as is the case with the non-ionic surfactant sodium dodecylbenzenesulfonate.

Preferred non-ionic surfactants are polyalkoxyalkanes, preferably a mixture of alkyl (C10-C20) ethoxylate with 15 to 35 EO or alkyl (C16-C18) ethoxylate with 15 to 35 EO and alkyl (C16-C18) ethoxylate with 25 EO, but also alkyl sulfonates such as sodium dodecylbenzenesulfonate or alkyl carbonates or alkyl sulfates are conceivable.

The use of a mixture of the non-ionic surfactants alkyl (C22) ethoxylate (25EO) and alkyl (C16-C18) ethoxylate (25EO) affects the cleaning ability. In addition, the joint use of these non-ionic surfactants makes the product easy to process.

By reducing the number of alkoxy groups, the non-ionic surfactants become more lipophilic, which can, for example, increase the solubility of perfume and thus the deodorizing intensity.

Molecules that generally act as thickeners in aqueous systems, such as hydrophilic substances, can also be used as non-ionic surfactants.

The concentration of the non-ionic surfactant to be used depends on its hydrophilicity and its network-forming ability. They can be, for example, 1 to 3 wt. %, 10 to 40 wt. %, preferably between 15 and 35 wt. % and particularly preferably between 20 and 30 wt. %, which is particularly preferred when using polyalkoxyalkanes.

In principle, all known anionic and/or non-ionic amphoteric surfactants can also be used as surfactants.

In addition to the components according to the invention, the sanitary product can comprise other conventional components, for example perfume oil, disinfectants, preservatives, such as isothiazolone derivatives or foam stabilizers, such as coconut fatty acid amidopropyl betaine or coconut fatty acid amidopropyl dimethylamine oxide, coconut fatty acid mono/diethanolamide or alkyl polyether glycerol ether sulfates, but also dyes and/or limescale- or urine scale-dissolving substances, in particular acids.

It is also possible to add olefin sulfonates, ether sulfates and/or surfactants, acid methyl taurides to the product according to the invention as foaming agents with a self-cleaning effect.

If desired, salts such as sodium sulfate can also be added to the formulation to increase the dissolution rate. The salt content can be up to 10 wt. %, preferably up to 5 wt. %.

The product according to the invention can be applied and renewed in a hygienic manner without touching potentially contaminated devices connected to the toilet bowl.

A significant advantage of the product according to the invention is that it can be portioned according to the consumer's wishes. If the consumer wants more intense deodorizing or if the toilet is used frequently, the dosage can be increased accordingly.

The product according to the invention can also be simultaneously applied in a simple manner to different places on the sanitary article, for example in order to achieve a uniform cleaning effect on both the right and left side of a toilet bowl.

The product according to the invention can also be applied in different compositions at different locations on a sanitary article. This makes it possible, for example, for two ingredients that are incompatible with one another, such as halogen-releasing agents and oxidation-sensitive perfumes, still to be used to jointly clean and/or deodorize the toilet by spatially separating them.

The adhesion to the sanitary object that is achieved, even when attached to a vertical surface, is so effective that the product is not removed even under the additional application of force by flushing water streams.

The sanitary products according to the invention can be rinsed off only after a relatively large number of flushes. The number of flushes depends naturally on the composition of the relevant sanitary product, the amount applied and the geometry of the sanitary product applied.

Fragrances and/or scents can also be added to the product to improve the air in the room, with the introduction of only fragrancing and scenting media into a toilet bowl in this way also being taken into consideration. The sanitary product can also be used for other sanitary articles such as urinals, pissoirs, but also for hand basins or the like.

Preferably, the product according to the invention is an ointment-like, pasty, non-gel-like, non-transparent product. The products are substantially dimensionally stable so that they do not “run down” or “drip”.

The products can preferably be introduced into the toilet bowl via tubes, similar to toothpaste tubes, syringes or cartridges, and continue to adhere thereto for a large number of consecutive flushes.

In addition to the components mentioned thus far, the WC cleaning block according to the invention can contain further ingredients that are usually used in WC cleaning blocks, preferably selected from the group comprising acids, bases, salts, thickening agents, antimicrobial active ingredients, preservatives, complexing agents, polymers, dyes, fragrances, perfume boosters, fillers, builders, bleaching agents, corrosion inhibitors, rinsing regulators, enzymes, microorganisms, active ingredients for inhibiting limescale deposits, active ingredients for reducing dirt adhesion, active ingredients for improving workability, active ingredients for reducing adhesiveness, and mixtures thereof. Overall, said cleaning block should not contain more than 60 wt. %, preferably 0.01 to 60 wt. %, in particular 0.2 to 15 wt. %, of further ingredients.

WC cleaning blocks according to the invention can contain one or more acids and/or the salts thereof in order to increase the cleaning performance on limescale and urine deposits. The acids are preferably produced from renewable raw materials. Therefore, in particular organic acids, such as formic acid, acetic acid, citric acid, glycolic acid, lactic acid, succinic acid, adipic acid, malic acid, tartaric acid and gluconic acid, and mixtures thereof, are suitable as acids. In addition, however, the inorganic acids hydrochloric acid, sulfuric acid, phosphoric acid and nitric acid, or also sulfamic acid or mixtures thereof, can also be used. The acids and/or salts thereof selected from the group comprising citric acid, lactic acid, formic acid, the salts thereof, and mixtures thereof are particularly preferred. Said acids and/or salts are used in amounts of 0.01 to 10 wt. %, particularly preferably 0.2 to 5 wt. %.

In a preferred embodiment, the agent in addition contains inorganic salts, preferably alkali- or alkaline-earth metal salts, in particular carbonates, sulfates, halides or phosphates, and mixtures thereof. Particularly preferably, sodium sulfate and/or sodium carbonate are used. Sodium sulfate can be contained in an amount of up to 60 wt. %, preferably 0.01 to 60 wt. %, particularly preferably 20 to 60 wt. %, in particular 35 to 55 wt. %. Sodium carbonate and further salts can be contained in an amount of up to 30 wt. %, preferably up to 10 wt. %, particularly preferably up to 5 wt. %.

Further alkalis can be contained in agents according to the invention. Bases from the group of alkali metal hydroxides and carbonates and alkaline earth metal hydroxides and carbonates, in particular sodium carbonate or sodium hydroxide, are preferably used as bases in products according to the invention. In addition, however, ammonia and/or alkanolamines having up to 9 C atoms in the molecule, preferably ethanolamines, in particular monoethanolamine, can also be used.

Disinfection and sanitation are a particular form of cleaning. In a corresponding particular embodiment of the invention, the WC cleaning block therefore contains one or more antimicrobial active ingredients, preferably in an amount of from 0.01 to 1 wt. %, preferably 0.02 to 0.8 wt. %, in particular 0.05 to 0.5 wt. %, particularly preferably 0.1 to 0.3 wt. %, extremely preferably 0.2 wt. %.

The terms “disinfection”, “sanitation”, “antimicrobial effect” and “antimicrobial active ingredient” have the conventional meaning thereof within the scope of the teaching according to the invention. While disinfection, in the narrower sense of medical practice, means killing—theoretically all—infectious germs, sanitation is to be understood as eliminating, as far as possible, all germs, including saprophytic germs that are usually harmless to humans. In this case, the extent of the disinfection or sanitation depends on the antimicrobial effect of the agent used, which effect reduces as the content of antimicrobial active ingredient decreases or as the agent to be used becomes increasingly diluted.

For example, antimicrobial active ingredients from the groups of alcohols, aldehydes, antimicrobial acids and the salts thereof, carboxylic acid esters, acid amides, phenols, phenol derivatives, diphenyls, diphenyl alkanes, urea derivatives, oxygen and nitrogen acetals and formals, benzamidines, isothiazoles and the derivatives thereof, such as isothiazolins and isothiazolinones, phthalimide derivatives, pyridine derivatives, antimicrobial surface-active compounds, guanidines, antimicrobial amphoteric compounds, quinolines, 1,2-dibromo-2,4-dicyanobutane, iodo-2-propynyl-butylcarbamate, iodine, iodophors, compounds which remove active chlorine, and peroxides are suitable according to the invention. Preferred antimicrobial active ingredients are preferably selected from the group comprising ethanol, n-propanol, i-propanol, 1,3-butanediol, phenoxyethanol, 1,2-propylene glycol, glycerol, undecylenic acid, citric acid, lactic acid, benzoic acid, salicylic acid, thymol, 2-Benzyl-4-chlorophenol, 2,2′-methylenebis(6-bromo-4-chlorophenol), 2,4,4′-trichloro-2′-hydroxydiphenyl ether, N-(4-chlorophenyl)-N′-(3,4-dichlorophenyl) urea, N,N′-(decane-1,10-diyldi-1-pyridyl-4-ylidene)bis(octylammonium) dichloride, N,N-bis(4-chlorophenyl) 3,12-diimino-2,4,11,13-tetraazatetradecandiimidamide, antimicrobial quaternary surface-active compounds, guanidines and sodium dichloroisocyanurate (DCI, 1,3-dichloro-5H-1,3,5-triazine-2,4,6-trione sodium salt). Preferred antimicrobially acting surface-active quaternary compounds contain an ammonium, sulfonium, phosphonium, iodonium or arsonium group. Furthermore, antimicrobially acting essential oils can also be used, which oils simultaneously fragrance the cleaning agent. Particularly preferred antimicrobial active ingredients are, however, selected from the group comprising salicylic acid, quaternary surfactants, in particular benzalkonium chloride, peroxo compounds, in particular hydrogen peroxide, alkali metal hypochlorite, sodium dichloroisocyanurate and mixtures thereof.

WC cleaning blocks according to the invention can also contain preservatives. The substances mentioned under the antimicrobial active ingredients can substantially be used as preservatives of this kind.

Complexing agents (INCI chelating agents), also referred to as sequestering agents, are ingredients which allow metal ions to complex and to become inactive in order to prevent disadvantageous effects of said ions on the stability or the appearance of the agent, for example cloudiness. It is important to complex the calcium and magnesium ions of the water hardness, which ions are incompatible with many ingredients. However, complexing ions of heavy metals, such as iron or copper, delays the oxidative decomposition of the finished agents. In addition, the complexing agents assist with the cleaning effect.

For example, the following complexing agents designated according to INCI are suitable: aminotrimethylene phosphonic acid, beta-alanine diacetic acid, calcium disodium EDTA, citric acid, cyclodextrin, cyclohexanediamine tetraacetic acid, diammonium citrate, diammonium EDTA, diethylenetriamine pentamethylene phosphonic acid, dipotassium EDTA, disodium azacycloheptane diphosphonate, disodium EDTA, disodium pyrophosphate, EDTA, etidronic acid, galactaric acid, gluconic acid, glucuronic acid, HEDTA, hydroxypropyl cyclodextrin, methyl cyclodextrin, pentapotassium triphosphate, pentasodium aminotrimethylene phosphonate, pentasodium ethylenediamine tetramethylene phosphonate, pentasodium pentetate, pentasodium triphosphate, pentetic acid, phytic acid, potassium citrate, potassium EDTMP, potassium gluconate, potassium polyphosphate, potassium trisphosphonomethylamine oxide, ribonic acid, sodium chitosan methylene phosphonate, sodium citrate, sodium diethylenetriamine pentamethylene phosphonate, sodium dihydroxyethylglycinate, sodium EDTMP, sodium gluceptate, sodium gluconate, sodium glycereth-1 polyphosphate, sodium hexametaphosphate, sodium metaphosphate, metasilicate, sodium phytate, sodium polydimethylglycinophenolsulfonate, sodium trimetaphosphate, TEA-EDTA, TEA-polyphosphate, tetrahydroxyethyl ethylenediamine, tetrahydroxypropyl ethylenediamine, tetrapotassium etidronate, tetrapotassium pyrophosphate, tetrasodium EDTA, tetrasodium etidronate, tetrasodium pyrophosphate, tripotassium EDTA, trisodium dicarboxymethyl alaninate, trisodium EDTA, trisodium HEDTA, trisodium NTA and trisodium phosphate.

The WC cleaning block according to the invention can additionally contain polymers. Said polymers can, for example, reduce calcification and the tendency to become resoiled.

Acrylic polymers, such as those which are commercially available from the company Rhodia under the trade name Mirapol, are preferred polymers.

The WC cleaning block according to the invention can contain one or more fragrances and/or one or more dyes (INCI colorants) as further ingredients. The dyes used can be water-soluble and oil-soluble dyes, with consideration needing to be taken to ensure compatibility with further ingredients, for example bleaching agents, and the dye used should not have a substantive effect on the WC ceramic, even after long-term use. The dyes are preferably contained in an amount of from 0.0001 to 0.1 wt. %, in particular 0.0005 to 0.05 wt. %, particularly preferably 0.001 to 0.01 wt. %.

Water-soluble and/or water-insoluble builders can optionally be used in the WC cleaning blocks according to the invention. Water-soluble builders are preferred since they are generally less likely to form insoluble residues on hard surfaces. Conventional builders which may be added within the scope of the invention are low-molecular-weight polycarboxylic acids and the salts thereof, homopolymeric and copolymeric polycarboxylic acids and the salts thereof, citric acid and the salts thereof, carbonates, phosphates and silicates. The category of water-insoluble builders includes zeolites, which may also be used, and mixtures of the aforementioned builders.

According to the invention, bleaching agents can be added to the cleaning agent. Suitable bleaching agents include peroxides, peroxy acids and/or perborates, hydrogen peroxide being particularly preferred. In contrast, sodium hypochlorite is less suitable in cleaning agents having an acidic formulation on account of the release of poisonous chlorine gas vapor, but can be used in alkaline cleaning agents. In some circumstances, a bleach activator may also be required in addition to the bleaching agent.

Suitable corrosion inhibitors (INCI) are, for example, the following substances named in accordance with INCI: cyclohexylamine, diammonium phosphate, dilithium oxalate, dimethylamino methylpropanol, dipotassium oxalate, dipotassium phosphate, disodium phosphate, disodium pyrophosphate, disodium tetrapropenyl succinate, hexoxyethyl diethylammonium, phosphate, nitromethane, potassium silicate, sodium aluminate, sodium hexametaphosphate, sodium metasilicate, sodium molybdate, sodium nitrite, sodium oxalate, sodium silicate, stearamidopropyl dimethicone, tetrapotassium pyrophosphate, tetrasodium pyrophosphate, and triisopropanolamine.

Viscosity regulators that include, for example, organic natural thickeners (agar-agar, carrageenan, tragacanth, gum arabic, alginates, pectins, polyoses, guar flour, carob seed flour, starch, dextrins, gelatin, casein), organic modified natural substances (carboxymethylcellulose and other cellulose ethers, hydroxyethyl cellulose and hydroxypropyl cellulose and the like, seed flour ethers), organic fully synthetic thickeners (polyacrylic and polymethacrylic compounds, vinyl polymers, polycarboxylic acids, polyethers, polyimines, polyamides), and inorganic thickeners (polysilic acids, phyllosilicates, clay minerals such as montmorillonites, zeolites, silicas), can optionally be used.

Preferred thickening agents are polysaccharides and heteropolysaccharides, in particular polysaccharide gums, for example gum arabic, agar, alginates, carrageenans and their salts, guar, guaran, tragacanth, gellan, ramsan, dextran or xanthan gum, and their derivatives, e.g., propoxylated guar, as well as mixtures thereof. Other polysaccharide thickeners, such as starches or cellulose derivatives, may be used alternatively to, but preferably in addition to, a polysaccharide gum, for example starches of various origins and starch derivatives, for example hydroxyethyl starch, starch phosphate esters or starch acetates, or carboxymethyl cellulose or the sodium salt thereof, methyl, ethyl, hydroxyethyl, hydroxypropyl, hydroxypropyl methyl or hydroxyethyl methyl cellulose or cellulose acetate. In addition to the thickeners mentioned, substances that are usually used as pressure-sensitive adhesives can also be used as adhesion promoters. These include, for example, adhesive polymers. However, it is important to ensure that the WC cleaning product is formulated in such a way that the bond between the cleaning product and the toilet bowl is not permanent but reversible, i.e. the adhesion forces are not too strong so that no sticky residues remain after the period of use has finished.

The substances referred to as rinsing regulators are used primarily to control the consumption of the agent during use such that the intended service life is met. Solid long-chain fatty acids, such as stearic acid, but also salts of such fatty acids, fatty acid ethanolamides, such as coconut fatty acid monoethanolamide, or solid polyethylene glycols, such as those having molecular weights of between 10,000 and 50,000, are preferably suitable as regulators.

The agent can also contain enzymes, preferably proteases, lipases, amylases, hydrolases and/or cellulases. They can be added to the agent according to the invention in any form established according to the prior art. These include solutions of the enzymes, advantageously as concentrated as possible, low in water and/or mixed with stabilizers. Alternatively the enzymes can also be encapsulated, for example by spray-drying or extruding the enzyme solution together with a preferably natural polymer or in the form of capsules, for example those in which the enzymes are enclosed in a solidified gel, or in the form of the core-shell type in which an enzyme-containing core is coated with a water-, air-, and/or chemical-impermeable protective layer. Further active ingredients, for example, stabilizers, emulsifiers, pigments, bleaches or dyes can additionally be applied in overlaid layers. Such capsules are made using methods that are known per se, for example by means of vibratory granulation or roll granulation or by means of fluid bed processes. Advantageously, such granules are low in dust, for example due to the application of polymeric film formers, and are stable in storage due to the coating.

Furthermore, enzyme stabilizers can be present in enzyme-containing products in order to protect an enzyme contained in a product according to the invention from damage, for example inactivation, denaturation, or decomposition caused, for example, by physical influences, oxidation, or proteolytic cleavage. Suitable in particular as enzyme stabilizers, each as a function of the enzyme used, are: benzamidine hydrochloride, borax, boric acid, boronic acid or the salts or esters thereof, in particular derivatives having aromatic groups, such as substituted phenylboronic acids or the salts or esters thereof; peptide aldehydes (oligopeptides having a reduced C-terminus), amino alcohols such as mono-, di-, triethanol-and-propanolamine and the mixtures thereof, aliphatic carboxylic acids up to C12, such as succinic acid, other dicarboxylic acids or salts of the mentioned acids; end-capped fatty acid amide alkoxylates; low aliphatic alcohols and in particular polyols, for example glycerol, ethylene glycol, propylene glycol or sorbitol; and reducing agents and antioxidants such as sodium sulfite and reducing sugars. Further suitable stabilizers are known from the prior art. Preferably, combinations of stabilizers are used, for example the combination of polyols, boric acid and/or borax, the combination of boric acid or borate, reducing salts and succinic acid or other dicarboxylic acids, or the combination of boric acid or borate with polyols or polyamino compounds and with reducing salts.

Various experiments were carried out. All quantities given indicate the amount of active ingredient in the composition in relation to the total amount.

Table 1 shows Composition C1:

| Component | C1 |
|---|---|
| Fragrances | 3 |
| APG, alkyl polyglucoside | — |
| LAS, alkylbenzene sulfonate | 18.7 |
| Sodium C14-16 olefin sulfonate | 9 |
| Trisodium citrate dihydrate | 0.7 |
| Fatty alcohol ethoxylate C16-C18 | 5 |
| 25 EO | |
| Sodium sulfate | 63.60 |

Composition C1 showed no adhesiveness whatsoever. Adhesion to smooth surfaces such as a toilet bowl was not possible.

Table 2 shows Compositions E1, E2 and E3 and the
results of the strength and adhesiveness measurements
carried out on these compositions:

| Component | E1 | E2 | E3 |
|---|---|---|---|
| Fragrances | 4 | 4 | 4 |
| APG, alkyl polyglucoside | 3 | 6 | 24 |
| LAS, alkylbenzene sulfonate | 22 | 22 | 22 |
| Sodium C14-16 olefin sulfonate | 11 | 11 | 11 |
| Trisodium citrate dihydrate | 0.7 | 0.7 | 0.7 |
| Fatty alcohol ethoxylate C16-C18 | 5 | 5 | 5 |
| 25 EO | | | |
| Sodium sulfate | 54.30 | 51.30 | 33.30 |
| Strength | 52.60 | 15.80 | 1.00 |
| Adhesiveness | 30.50 | 13.20 | 0.90 |

Unlike composition C1, compositions E1, E2 and E3 are self-adhesive compositions capable of adhering to the sanitary article independently. Unlike the compositions known from the prior art, the compositions described are not solid gels. Rather, they are stable molded body masses, which are preferred over solid gels. Surprisingly, it was found that these molded bodies are able to adhere independently to the sanitary article, which can be attributed to the presence of APG. Of the experiments shown, Examples E1 and E2 are preferred over E3. E3 was able only to show adhesion to the surface. According to a preferred embodiment, APG is therefore present in amounts of at most 24 wt. %, preferably in an amount of less than 24 wt. %. According to another preferred embodiment, APG is present in amounts of at least 1 wt. % to less than 24 wt. %. E2 is in turn preferred over E1 because it has more balanced strength and adhesiveness values, making it more suitable for use on a toilet surface. According to a further preferred embodiment, APG is present in amounts of at least 4 wt. % to less than 24 wt. %, more preferably at least 4 wt. % to less than 20 wt. %.

Table 3 shows the particularly preferred compositions,
E4, E5 and E6, and the results of the strength and adhesiveness
measurements carried out on these compositions:

| Component | E4 | E5 | E6 |
|---|---|---|---|
| Fragrances | 4 | 4 | 4 |
| APG, alkyl polyglucoside | 10 | 12 | 14 |
| LAS, alkylbenzene | 22 | 22 | 22 |
| sulfonate | | | |
| Sodium C14-16 olefin | 11 | 11 | 11 |
| sulfonate | | | |
| Trisodium citrate dihydrate | 0.7 | 0.7 | 0.7 |
| Fatty alcohol ethoxylate | 5 | 5 | 5 |
| C16-C18 25 EO | | | |
| Sodium sulfate | 47.30 | 45.30 | 43.30 |
| Strength | 10.50 | 9.50 | 6.80 |
| Adhesiveness | 8.90 | 7.60 | 6.10 |

Compositions E4, E5 and E6 are particularly preferred since the results of the strength and adhesiveness measurements carried out on these compositions show particularly balanced values. The compositions adhere particularly well to surfaces and show a good consistency (does not adhere too strongly) and adequate rinseability. Compositions E4, E5 and E6 are therefore stable molded body masses, which are preferred over solid gels. Surprisingly, it has been found that these molded bodies are able to independently adhere to the sanitary article and at the same time have a very good consistency, which can be attributed to the presence of APG, in particular in preferred amounts of at least 7 wt. % to less than 24 wt. %, more preferably 9 to less than 24 wt. % and even more preferably 9 to 17 wt. %, and particularly preferably 9 to 15 wt. %.

The presence of anionic surfactant (LAS) contributes to better foaming and improved cleaning performance.

The presence of olefin sulfonate contributes to better formability. In this respect, olefin sulfonate advantageously works with the APG, which provides the adhesion capability and consistency of the composition.

The strength (also called hardness) and adhesiveness were determined experimentally:

The hardness and adhesiveness measurements were carried out by ball pressure testing. A TA.XTPlus 100 Connect was used for measuring hardness and adhesiveness. The penetration depth for the steel ball to be inserted into the sample was set at 5 mm. The steel ball was then inserted into the sample at a constant speed of 0.1 mm/s and the force required therefor was measured to determine the strength. The force that had to be applied to pull the ball back out of the sample corresponded to the adhesiveness measurement.

1. A self-adhesive sanitary product comprising alkyl polyglycoside.

2. The self-adhesive sanitary product of claim 1, wherein the self-adhesive sanitary product is not a solid gel.

3. The self-adhesive sanitary product of claim 1, wherein the alkyl polyglycoside is present in an amount of at most 24 wt. %, based on the total weight of the self-adhesive sanitary product.

4. The self-adhesive sanitary product of claim 1, wherein the alkyl polyglycoside is present in an amount of at least 2 wt. %, based on the total weight of the self-adhesive sanitary product.

5. The self-adhesive sanitary product of claim 1, wherein the alkyl polyglycoside is present in an amount of from 1 wt. % to less than 24 wt. %, based on the total weight of the self-adhesive sanitary product.

6. The self-adhesive sanitary product of claim 1, further comprising at least one anionic surfactant.

7. The self-adhesive sanitary product of claim 6, wherein the at least one anionic surfactant comprises an olefin sulfonate.

8. The self-adhesive sanitary product of claim 7, wherein the olefin sulfonate is present in an amount of from 2 wt. % to 30 wt. %, based on the total weight of the self-adhesive sanitary product.

9. The self-adhesive sanitary product of claim 6, further comprising at least one further anionic surfactant.

10. The self-adhesive sanitary product of claim 9, wherein the at least one further anionic surfactant is an alkylbenzene sulfonate.

11. The self-adhesive sanitary product of claim 10, wherein the alkylbenzene sulfonate is present in an amount of from at least 5 wt. % to 40 wt. %, based on the total weight of the self-adhesive sanitary product.

12. The self-adhesive sanitary product of claim 1, further comprising at least one non-ionic surfactant.

13. The self-adhesive sanitary product of claim 12, wherein the at least one non-ionic surfactant is a fatty alcohol ethoxylate.

14. The self-adhesive sanitary product of claim 19, wherein the alkylbenzene sulfonate is present in an amount of from 2 wt. % to 10%, based on the total weight of the self-adhesive sanitary product.

15. A method comprising applying the self-adhesive sanitary product of claim 1 to a surface of a sanitary article.

16. The self-adhesive sanitary product of claim 5, wherein the alkyl polyglycoside is present in an amount of from 2 to less than 24 wt. %, based on the total weight of the self-adhesive sanitary product.

17. The self-adhesive sanitary product of claim 16, wherein the alkyl polyglycoside is present in an amount of from 4 wt. % to 20 wt. %, based on the total weight of the self-adhesive sanitary product.

18. The self-adhesive sanitary product of claim 17, wherein the alkyl polyglycoside is present in an amount of from 9 wt. % to 15 wt. %, based on the total weight of the self-adhesive sanitary product.

19. The self-adhesive sanitary product of claim 8, wherein the olefin sulfonate is present in an amount of from at least 7 wt. % to 15 wt. %, based on the total weight of the self-adhesive sanitary product.

20. The self-adhesive sanitary product of claim 11, wherein the alkylbenzene sulfonate is present in an amount of from 10 wt. % to 30 wt. %, based on the total weight of the self-adhesive sanitary product.
